(19) United States
(12) Patent Application Publication
(10) Pub. No.: US 2026/0192076 A1
(43) Pub. Date: Jul. 9, 2026

(54) Installation for administering gaseous NO with gas injection module

(71) Applicant: INOSYSTEMS, ANTONY (FR)

(72) Inventors: Thierry BOULANGER, ANTONY (FR); Akshar PATEL, ANTONY (FR); Jiefu MA, ANTONY (FR); Frederic MARCHAL, ANTONY (FR)

(21) Appl. No.: 19/415,024

(22) Filed: Dec. 10, 2025

(30) Foreign Application Priority Data
Jan. 6, 2025 (FR) 2500093

(51) Int. Cl. A61M 16/08

(52) U.S. Cl. CPC A61M 16/0875; A61M 2202/0208; A61M 2202/0275

(57) ABSTRACT

The invention relates to a gas supply installation (1, 2, 3) comprising a gas injection module (500, 800), to which there are connected a mechanical ventilator (2) and an NO delivery apparatus (1). The gas injection module (500, 800) comprises a tubular module body (803) through which there extends an internal passage (811) of longitudinal axis (AA) and which comprises an upstream port (801); a downstream port (802); an external injection conduit (807) connecting to the tubular module body (803) and comprising an internal passage (811) for conveying an NO/N₂ gas flow; and a tubular expansion (812) forming an injection nozzle arranged in the internal passage (811) and comprising an injection port (809) arranged at a free end (812.1) and opening into the internal passage (811) of the tubular module body (803). The tubular expansion (812) has a generally curved or angled shape and comprises a section carrying the injection port (809), arranged parallel to the axis (AA) and directed towards the downstream port (802).

### CROSS-REFERENCE TO RELATED APPLICATIONS

This patent application claims priority to French Application No. 2500093, filed Jan. 6, 2025 and the entire contents of which are incorporated herein by reference.

### BACKGROUND

The invention relates to an installation for supplying NO-based gas to a person in need thereof for the purpose of assisting their respiratory functions, typically a patient, comprising an improved gas injection module arranged in the patient circuit of the installation for conveying a flow of respiratory gas containing oxygen (>20 vol %) from a medical ventilator, such as an N₂/O₂ mixture or air, said injection module being configured for further connection thereto of an NO delivery apparatus supplying an NO-containing gas flow, so as to be able to effect homogeneous mixing of these gases by means of said injection module and thereby obtain a combined gas mixture containing NO at a given dosage, which is then administered by inhalation to the patient in need thereof.

Nitric oxide or NO is a gas which, when inhaled, dilates the pulmonary blood vessels, thereby making it possible to increase oxygenation of the blood by improving gas exchange. This property is exploited for treating pulmonary arterial hypertension in adults, children or neonates.

Usually, NO, typically a mixture of NO and nitrogen (N₂), is added to the non-hypoxic respiratory gas, which is inhaled by the patient with pulmonary hypertension at a specified dosage, usually set by a physician, which typically ranges between 1 and 100 ppm by volume, typically between 1 and 40 ppm vol.

For this purpose, the NO/N₂ mixture is supplied by an NO delivery apparatus which fluidically connects to an NO injection module arranged in the inspiratory branch of a patient circuit of an installation for supplying gaseous NO, i.e. an NO-based gas mixture, supplying the patient to be treated.

The patient circuit serves to fluidically connect a medical ventilator, that is to say a respiratory assistance apparatus, supplying the respiratory gas containing at least 20 to 21 vol % of oxygen, such as air or an O₂/N₂ mixture, to a respiratory interface supplying the patient to be treated, such as a breathing mask, a tracheal tube or the like, as taught by U.S. Pat. No. 5,558,083 or EP4209243.

The flow of respiratory gas (e.g. air or O₂/N₂) from the medical ventilator is therefore conveyed via the patient circuit and passes through the NO injection module where it is mixed with the NO/N₂ mixture, and the combined gas mixture exiting the NO injection module travels through the patient circuit to the respiratory interface supplying the combined NO-containing mixture to the patient.

In other words, the NO injection module allows the NO/N₂ mixture from the NO delivery apparatus to be injected into the flow of respiratory gas coming from the medical ventilator, in order thereby to obtain the combined or final gaseous mixture containing at least N₂, O₂ (>20 vol %) and NO at the desired dosage.

The quantity of NO/N₂ mixture, i.e. the flow rate of NO/N₂, to be injected into the flow of respiratory gas via the NO injection module is calculated on the basis of flow rate measurements performed by a flow rate sensor arranged in the patient circuit between the medical ventilator and said NO injection module. The flow rate sensor serves to measure the flow rate of the respiratory gas flow delivered by the medical ventilator and to return the measurements to the NO delivery apparatus so that the latter can deduce therefrom, i.e. calculate, the flow rate of NO to be delivered, i.e. of NO/N₂ gas mixture, in relation to the desired dosage. The flow rate sensor can be associated with or integrated into the NO injection module.

In practice, there is a problem resulting from injection of the flow of NO via the NO injection module into the oxygen-containing respiratory gas from the medical ventilator, namely an excessive oxidation of the NO, within the NO injection module, by oxygen (O₂) present in the flow of respiratory gas, with formation of toxic nitrogen dioxide (NO₂) species in the combined gas mixture sent to the patient.

While this untimely oxidation of NO, with formation of NO₂, depends on the contents of NO and O₂, it is all the more important the higher the NO content of the NO/N₂ mixture. Indeed, the formation of NO₂ depends on the initial NO concentration squared, whereas it is linear with the O₂ concentration. In other words, the initial concentration of NO has a much greater impact than that of O₂ on the formation of NO₂.

However, the current NO injection modules have been designed for initial NO concentrations in the NO/N₂ mixture of less than 800 ppmv of NO and therefore cannot guarantee an acceptable NO₂ concentration when the NO concentration is higher, for example by several thousand ppmv. As a guide, changing from an NO content of 800 ppmv to an NO content of 5000 ppmv in the NO/N₂ mixture supplied by the NO delivery apparatus leads to multiplying the NO concentration by 6.25, which results in a 39 times greater formation of NO₂. Similarly, changing from an NO content of 800 ppmv to 20000 ppmv leads to a 625 times greater formation of NO₂.

A homogeneous combined gas mixture at the desired NO dosage, for example 20 ppmv NO, should therefore be able to be obtained as quickly as possible, because when it is homogeneous, the oxidation of NO is slower and the formation of NO₂ is lower, even with high contents of NO, typically of several thousand ppmv.

To this end, U.S. Pat. No. 10,894,140 proposes using an NO injection module with a single injection port or multiple injection ports, i.e. one or more NO inlets, considering a ratio not to be exceeded in terms of gas velocity, that is to say the ratio between the velocity of respiratory gas (O₂>20 vol %) from the medical ventilator and that of the NO-based gas from the NO delivery device (i.e. N₂/NO mixture). In both cases, the injection is effected transversely where the two gases mix with each other in the injection module by being directed/oriented perpendicularly to each other.

However, using an NO injection module designed for transverse injection is not ideal. Indeed, it has been found that O₂ present in the flow coming from the ventilator can diffuse up into the gas line connecting the NO delivery apparatus to the NO injection module during the operation of the installation and can thus generate an additional quantity of NO₂ which will then be found in the combined mixture leaving the NO injection module. In addition, it has also been observed that transverse injection of NO can locally create an imbalance of concentrations and can lead to excessive formation of NO₂. These additional formations of NO₂ species are neither desirable nor acceptable, especially the higher the concentration of NO in the supplied flow (i.e. NO/N₂).

In general, because of the accelerated formation of NO₂ as a function of the NO concentration of the N₂/NO source, it appears important to further limit the formation of NO₂, in particular for high concentrations of the N₂/NO source.

An object of the invention is therefore to propose an installation for supplying NO-based gas comprising a patient circuit, in which an improved NO injection module is arranged, making it possible to minimize the formation of toxic NO₂ from oxidation of NO, while leading to the formation of a homogeneous combined gas mixture, after injection, by means of this module, of an NO-based gas flow (e.g. NO/N₂) from an NO delivery apparatus into a respiratory gas flow containing oxygen (e.g. air or O₂/N₂) from a medical ventilator, preferably even when the NO content in the flow of NO (e.g. NO/N₂) is high, i.e. typically several thousand ppmv.

### SUMMARY

A solution according to the invention relates to an installation for supplying gas, typically a combined gas mixture containing NO and oxygen, comprising a gas injection module to which there are connected a mechanical ventilator, supplying a flow of respiratory gas containing at least 20 vol % of oxygen, and an NO delivery apparatus, supplying a flow of an NO/N₂ gas mixture.

The gas injection module comprises a tubular module body through which there extends an internal passage (i.e. lumen) having a longitudinal axis (AA).

The tubular module body further comprises:
an upstream port (i.e. inlet orifice or similar) through which the flow of respiratory gas can enter the internal passage,
a downstream port (i.e. outlet orifice or the like) through which a combined gas flow can exit the internal passage,
an external injection conduit fluidically connecting to the tubular module body and comprising an internal passage for conveying the flow of NO/N₂ gas mixture, and
a tubular expansion forming an injection nozzle arranged in the internal passage of the tubular body and comprising an internal nozzle passage in fluidic communication with the internal passage of the injection conduit, said tubular expansion further comprising an injection port arranged at a free end of said tubular expansion, said injection port opening into the internal passage of the tubular module body.

Moreover, in the gas injection module of the installation of the invention, the tubular expansion has a generally curved or angled shape, that is to say it forms a “curved nozzle”, and comprises at least one section carrying the injection port, arranged parallel to the longitudinal axis (AA) and directed (i.e. oriented) towards the downstream port.

Thus, by virtue of the gas injection module with curved nozzle used in the installation according to the invention, the NO/N₂ mixture is injected into the internal passage (i.e. lumen) of longitudinal axis (AA) of the module in the same direction, that is to say along the axis AA, as the flow of respiratory gas containing at least 20 vol % of oxygen, entering through the upstream port and flowing through the internal passage (lumen) of the tubular body towards the downstream port through which the combined gas flow, i.e. the gas flow containing NO and oxygen, exits.

Depending on the embodiment in question, the gas injection module of the installation according to the invention can comprise one or more of the following features:
the upstream port of the gas injection module is fluidically connected to the mechanical ventilator supplying the flow of respiratory gas, preferably via at least a part of the inspiratory branch of the patient circuit.
the external injection conduit is fluidically connected to the NO delivery apparatus, preferably via a gas conduit, such as a flexible hose.
the internal passage comprises an upstream chamber comprising the upstream port, i.e. the upstream port fluidically communicates with the upstream chamber.
the internal passage comprises a downstream mixing chamber comprising the downstream port, i.e. the downstream port fluidically communicates with the downstream mixing chamber.
the tubular module body is preferably cylindrical or similar.
the tubular expansion has the general shape of an “L” or similar.
the section carrying the injection port of the tubular expansion is arranged coaxially with respect to the longitudinal axis (AA) of the internal passage of the tubular module body.
the tubular expansion comprises a first conduit section and a second conduit section.
the second conduit section comprises the injection port and the free end of said tubular expansion.
the first conduit section is arranged between the second conduit section and the internal passage of the injection conduit and in fluidic communication with the second conduit section and the internal passage.
the external injection conduit connects to the tubular peripheral wall of the tubular module body.
the tubular module body is preferably made of polymer, typically of polycarbonate or polysulfone for example.
the tubular expansion is formed in one piece, for example by injection moulding or any other suitable technique.
the tubular expansion constitutes an angled, i.e. curved, injection nozzle.
the tubular module body is configured, at the upstream port and the downstream port, to permit a fluidic connection of gas conduits forming at least a part of the inspiratory branch of the patient circuit, typically an upstream gas conduit and a downstream gas conduit, such as flexible hoses.
the tubular module body comprises conduit connection means for fluidically connecting thereto the upstream and downstream gas conduits of the inspiratory branch of the patient circuit.
the conduit connection means comprise couplings or the like, for example couplings of the plug-in, screw-in or bayonet type or similar.
the upstream gas conduit constitutes an upstream part of the inspiratory branch of the patient circuit for fluidically connecting the medical ventilator to the gas injection module in order to feed it with respiratory gas containing at least 20 vol % of O₂.
the patient circuit feeds a combined gas mixture to a respiratory interface on the patient.
the respiratory interface comprises a breathing mask, tracheal intubation tube or other suitable breathing device.
the downstream gas conduit constitutes a downstream part of the inspiratory branch of the patient circuit used to collect and convey the combined gas mixture formed within the tubular module body, i.e. to fluidically connect the gas injection module to the respiratory interface on the patient.
the tubular expansion of the gas injection module is oriented to inject the NO/N₂ mixture into the internal passage of longitudinal axis (AA) in the same direction as the flow of respiratory gas containing at least 20 vol % of oxygen flowing through the internal passage, i.e. in the same direction of flow as the respiratory gas flow and along the axis (AA).

In addition, according to some embodiments, the gas supply installation according to the invention can also comprise one or more of the following additional features:
the NO delivery apparatus is fluidically connected and fed with an NO/N₂ mixture by one or more gas containers containing an NO/N₂ mixture, such as gas cylinders.
the NO delivery apparatus comprises operating means, preferably microprocessor-based, such as an electronic controller or the like; an internal gas circuit comprising at least one NO inlet configured for fluidically connecting thereto at least one NO container; valve means, such as solenoid valves, arranged on the internal gas circuit for controlling the flow of NO/N₂ in the internal gas circuit; and at least one NO outlet for supplying the NO/N₂ gas flow.
expansion means, such as one or more gas expansion devices, are arranged upstream of the NO delivery apparatus and are configured to reduce the pressure of the NO-containing gas, coming from the one or more NO containers, to a given expansion pressure (PD) of less than 10 bar, typically of the order of 4 to 8 bar, preferably between 4 and 7 bar.
at least one gas feed line for conveying the NO-containing gas, which has passed through the expansion means, to said one or more gas inlets of the NO delivery apparatus, for example one or more flexible hoses or the like.
when the one or more NO containers are full, the NO/N₂ mixture contained therein is at a pressure (i.e. a starting pressure) of at least 150 bar, referred to as “high pressure”. This high pressure corresponds to the pressure of the NO-based gas before expansion, e.g. the NO/N₂ mixture, that is to say before pressure reduction by passage through the first and/or second pressure-reducing means.
each gas container is equipped with a gas distribution valve serving to control the supply of NO-based gas from said gas container, i.e. the distribution of gas.
each NO container contains an NO/N₂ mixture containing between 100 and 20000 ppmv.
the NO delivery apparatus is fed with an NO/N₂ mixture containing between 100 and 20000 ppmv of NO, the remainder being nitrogen (N₂).
the NO delivery apparatus comprises dose adjustment means which are configured to allow a user to set or select an NO content setpoint corresponding to the final proportion of NO required in the combined gaseous mixture, i.e. a dosage.
the dose adjustment means form part of an HMI (human-machine interface) or GUI (graphical user interface) of the NO delivery apparatus.
the dose adjustment means of the apparatus comprise one or more touch keys which can be actuated by the user and are displayed on a digital touch screen of the HMI.
the touch screen is preferably of the type displaying in colour.
the NO content setpoint is between 1 and 80 ppmv, typically between 5 and 40 ppmv.
the storage means of the delivery apparatus comprise a computer memory, such as a flash memory, a RAM or the like.
the operating means comprise one or more (micro) processors arranged on one or more electronic boards, typically a (micro) controller or the like.
the one or more (micro) processors implement one or more algorithms, in particular one or more algorithms for operating or controlling valves, for processing flow rate or pressure measurements, etc.
the storage means are arranged on the electronic board.
the NO delivery apparatus and the mechanical ventilator are each powered electrically by one or more electric current sources, typically the mains supply (110/220 V) and/or one or more rechargeable batteries.
the mechanical ventilator is configured to supply a flow of respiratory gas containing 02 in a content of at least 20 vol %, such as air or an O₂/N₂ mixture, typically of at least 21 vol % approximately.
the NO delivery apparatus and the mechanical ventilator are fluidically connected to the injection module arranged on/in the patient circuit, in particular on/in the inspiratory branch of the patient circuit.
the injection module is configured to mix the flow of NO-based gas from the NO delivery apparatus (i.e. the NO/N₂ gas flow) with the flow of respiratory gas containing at least 20 vol % of O₂ supplied by the ventilator, and to obtain a combined gas mixture containing (at least) NO and oxygen.
a flow rate sensor is arranged in the patient circuit between the medical ventilator and the injection device, in particular in the inspiratory branch of the patient circuit.
the injection module comprises a first gas inlet fed with a flow of respiratory gas containing O₂ from the medical ventilator; and a second gas inlet fed with NO-containing gas from the NO delivery apparatus.
the injection module also comprises a combined gas outlet supplying the combined gas mixture containing NO and oxygen, which mixture is obtained by mixing, within the injection module, the NO-containing gas (e.g. the NO/N₂ mixture) with the flow of respiratory gas containing O₂ (e.g. air or O₂/N₂ mixture).
the medical ventilator comprises a motorized blower (i.e. turbine, compressor or similar) delivering the respiratory gas, typically air or an oxygen/nitrogen mixture or, according to another embodiment, an internal gas circuit comprising one or more proportional valves for conveying the gas and controlling its supply, in particular its flow rate. Such a ventilator is generally supplied with respiratory gas via one or more wall outlets supplied with gas from a network of channels in a hospital establishment or building, typically with air or an oxygen/nitrogen mixture.
the medical ventilator comprises control means or a control device, such as one or more electronic control boards. Preferably, the control means of the medical ventilator operate or control the motorized blower or, depending on the circumstances, the proportional valves of the medical ventilator.
according to a particular embodiment, the medical ventilator is of the HFO type or comprises an HFO function, that is to say it is able to produce high-frequency oscillations.
each NO container is or comprises one or more gas cylinders with a capacity of between 0.5 and 50 l (water equivalent).
each NO container comprises a cylindrical body made of steel or of aluminium alloy.
the patient circuit of the installation comprises an inspiratory branch and an expiratory branch, typically flexible conduits forming the inspiratory branch and the expiratory branch, for example hoses made of polymer.
the inspiratory branch and the expiratory branch, e.g. flexible conduits, are connected at a joining piece, such as a Y-piece.
the inspiratory branch and/or the expiratory branch are fluidically connected to a patient respiratory interface, preferably via the joining piece.
the patient circuit, also called the respiratory circuit, in particular the inspiratory branch, can comprise a gas humidifier, preferably arranged downstream of the injection module, so as to be able to humidify the combined gas mixture before it is administered by inhalation to the patient.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstrictions or similar, said method comprising administration by inhalation, to the person requiring it, of a gaseous mixture containing from 1 to 80 ppmv of NO and at least 20 vol % of oxygen approximately, preferably at least 21 vol % of oxygen approximately, by means of a gas supply installation, as described above according to the invention, comprising an NO delivery apparatus for delivering NO at the required dosage, so as to treat (at least partially) said pulmonary hypertension and/or said hypoxia, which can be caused by one or more pulmonary diseases or disorders such as PPHN (persistent pulmonary hypertension of the newborn) or ARDS (acute respiratory distress syndrome) or can be caused by heart surgery with placement of the patient on extracorporeal blood circulation (ECC).

Moreover, the invention also relates to the use of a gas injection module to fluidically connect a mechanical ventilator, supplying a flow of respiratory gas containing at least 20 vol % of oxygen, and an NO delivery apparatus, supplying a flow of an NO/N₂ mixture from a gas supply installation to a patient, in such a way as to mix said gas flows and obtain a combined mixture containing NO, oxygen (i.e. at least 20 vol % of O₂) and nitrogen (N₂).

### Definitions

In general, within the context of the invention:
“ppmv” means parts per million by volume.
“vol % means percentage by volume.
“NO” denotes nitric oxide.
“NO₂” denotes nitrogen dioxide.
“N₂” denotes nitrogen.
“O₂” denotes oxygen.
the pressures are expressed in bar absolute, abbreviated to “bar”.
the terms “concentration”, “quantity”, “proportion”, “dose” and “content” are considered as equivalents.
the terms “means of/to/for” are considered to be wholly equivalent to and capable of being substituted by the terms “device of/to/for”, for example the term “control means” may be replaced by “control device”, the term “valve means” may be replaced by “valve device”, the “storage means” may be replaced by “storage device”, etc.
“pressure measurement” is understood as a pressure value (e.g. a numerical value) or a signal representative of such a pressure value, which reflects or corresponds to the gaseous pressure measured by a pressure sensor or the like, or any other suitable sensor.
“flow rate measurement” is understood as a flow rate value (e.g. a numerical value) or a signal representative of such a flow rate value, which reflects or corresponds to a gas flow rate measured by a flow rate sensor, or the like, or any other suitable sensor.
the terms “upstream” and “downstream” are used in relation to the normal direction of flow of the gas in question, for example in the direction from the gas container(s) to the delivery apparatus and then to the patient for the NO/N₂ mixture, or in the direction from the ventilator to the injection module and then to the patient for the respiratory gas containing oxygen (i.e. >20 vol %).

### BRIEF DESCRIPTION OF VIEWS OF THE DRAWINGS

The invention will now be better understood from the following detailed description, provided by way of non-limiting illustration, with reference to the appended figures, in which:

FIG. 1 shows schematically an embodiment of an NO delivery installation.

FIG. 2 shows an embodiment of an injection module according to the prior art.

FIG. 3 and FIG. 4 show an embodiment of another injection module according to the prior art.

FIG. 5 and FIG. 6 show schematically an embodiment of an injection module for an installation according to the present invention, such as that in FIG. 1.

FIG. 7 is a graph illustrating the phenomenon of gas diffusion in the injection modules of FIG. 2 to FIG. 6.

FIG. 8 shows schematically an embodiment of an experimental set-up used in comparative tests.

### DETAILED DESCRIPTION

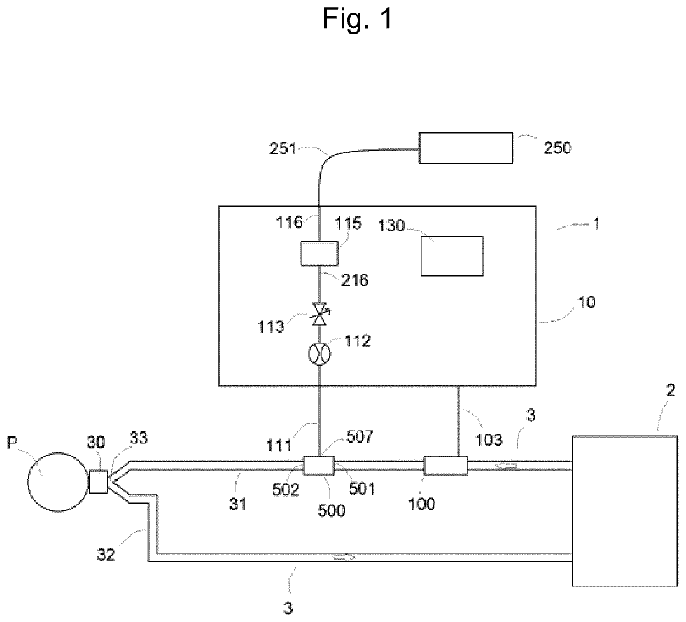

FIG. 1 shows schematically an embodiment of a gas supply or delivery installation 1, 2 according to the present invention, comprising an NO delivery device or apparatus 1 and a mechanical ventilator 2, also called a medical ventilator, that is to say a respiratory assistance apparatus delivering a non-hypoxic oxygen-based respiratory gas, feeding a patient circuit 3, in particular the inspiratory branch 31 of said patient circuit 3, so as to obtain a combined gas mixture, also called a final gas mixture, intended to be subsequently administered by inhalation to a patient P to be treated, that is to say a combined gas mixture containing oxygen (>20 vol %, preferably >21 vol %), nitrogen and a desired concentration of NO corresponding to a dosage set by an anaesthetist or the like, typically between 1 and 80 ppmv of NO.

The NO delivery apparatus 1 is fed with gaseous NO, typically as a gaseous mixture NO/N₂, coming from one or more NO sources 250 connected fluidically to the NO delivery apparatus 1, in particular to a high-pressure line 116 of the NO delivery apparatus 1, via a supply line 251, such as a flexible conduit or similar.

Typically, the NO source 250 comprises one or more pressurized gas cylinders holding an NO/N₂ mixture containing a given concentration of NO, conditioned at a pressure (when completely full, i.e. before any withdrawal) of up to 140 to 200 bar abs, or more.

Depending on the case, the NO/N₂ mixture can comprise an NO concentration or content: either what is called “conventional”, namely between 100 and 1000 ppmv, for example of the order of 400 to 800 ppmv, or what is called “high”, namely greater than 1000 ppmv, typically greater than 2000 ppmv, for example from 5000 to 20,000 ppmv.

The NO delivery apparatus 1 makes it possible to supply the patient circuit 3 with NO in gaseous form, typically the NO/N₂ gas mixture containing the “conventional” or “high” NO content, while the medical ventilator 2 delivers a non-hypoxic respiratory gas containing at least 20 vol % of oxygen, preferably at least 21 vol % of oxygen, such as air or an N₂/O₂ mixture, in the patient circuit 3.

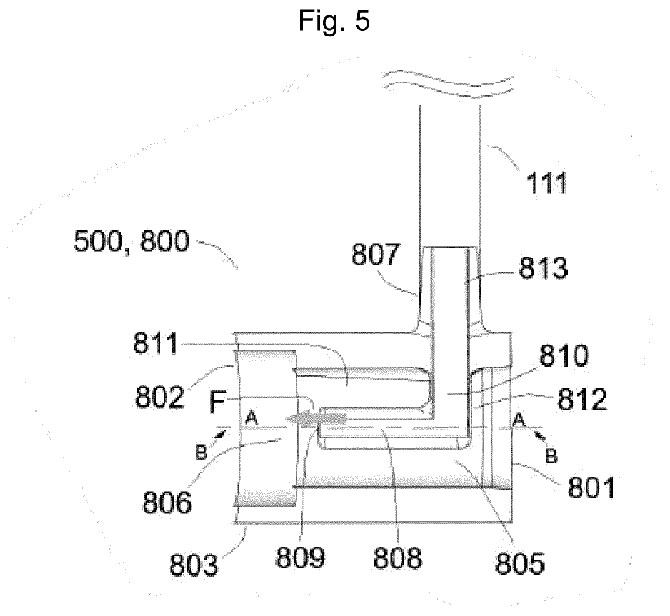

In order to permit homogeneous mixing between the NO-containing gas flow, i.e. the NO/N₂ mixture coming from the NO delivery apparatus 1, and the oxygen-containing respiratory gas flow coming from the medical ventilator 2, such as air or an O₂/N₂ mixture, and to thus obtain the desired combined mixture, an NO injection module 500 is arranged in the inspiratory branch 31 of the patient circuit 3, to which the NO delivery apparatus 1 is connected via an injection line 111 which is connected to an NO/N₂ injection channel 507 of the NO injection module 500, as detailed below, in particular the NO injection module 500, 800 of the invention illustrated in FIG. 5 as explained below.

Specifically, the NO injection module 500 also comprises:
a respiratory gas inlet, also called an upstream port, to which there is fluidically connected an upstream gas conduit forming an upstream part of the inspiratory branch 31, which conveys the respiratory gas coming from the medical ventilator,
an NO/N₂ mixture injection channel 507 fluidically connected to the injection line 111, and
a combined gas outlet, also called a downstream port, to which there is fluidically connected a downstream gas conduit forming a downstream part of the inspiratory branch 31, which serves to collect the combined gas mixture (i.e. NO, N₂, O₂) formed within the NO injection module 500 and to then convey it to the patient.

In order to be able to fluidically connect to it the upstream and downstream gas conduits of the inspiratory branch 31 of the patient circuit, and/or the injection line 111, the tubular module body 500 comprises suitable conduit connection means, such as couplings or the like, for example of the plug-in, screwed or bayonet type or the like.

The inspiratory branch 31 and expiratory branch 32 of the patient circuit 3 generally comprise gas conduits, also called channels, hoses, passages, tubes or similar, for example flexible polymer hoses that are able and configured to convey the gas flows.

The inspiratory branch 31 then makes it possible to convey and deliver the combined gas mixture containing NO to the patient P, during the inspiratory phases of the latter, hence to provide respiratory assistance to the patient P, while the expiratory branch 32 makes it possible to convey the gases exhaled by the patient, during the expiratory phases of the latter.

As can be seen in FIG. 1, the inspiratory branch 31 and expiratory branch 32 are fluidically connected to a junction piece 33, such as a Y-piece or the like, in fluidic communication with a respiratory interface 30 for delivering the combined gas to patient P and for collecting the exhaled gases rich in CO₂ and delivering them to the medical ventilator 2 before they are discharged to the atmosphere. The respiratory interface 30 can be a face mask, an intubation tube or any other suitable administration device.

For its part, the medical ventilator 2 is a conventional respiratory assistance apparatus or similar, comprising for example a motorized (micro) blower, also called a turbine or compressor, which delivers the respiratory gas (e.g. air or N₂/O₂) into the patient circuit 3 and of which the operation is controlled by one or more electronic control boards or similar.

Both the ventilator 2 and the NO delivery apparatus 1 are electrically powered by power supply means (not shown), such as the mains (110/220V) and/or an internal battery or batteries.

Furthermore, a flow rate sensor 100 and the NO injection module 500 are arranged in the inspiratory branch 31 of the patient circuit 3, the flow rate sensor 100 preferably being arranged between the NO injection module 500 and the mechanical ventilator 2.

The flow rate sensor 100 can be a mass sensor or a differential pressure sensor, preferably a mass flow rate sensor as illustrated in FIG. 1, for example that of the company Sensirion® designated SFM3300-D. The flow rate sensor 100 is used to measure the gas flow, i.e. a flow rate, delivered by the medical ventilator 2 in the upstream part of the inspiratory branch 31. To do this, the sensor 100 can be connected electrically, for example via an electric cable 103, to the operating unit 130 of the NO delivery apparatus 1 or can transmit the flow rate measurements thereto so that they are processed there by computer.

The operating unit 130 comprises a system for processing data, i.e. measurements, which is arranged in the shell 10 of the apparatus 1 and comprises one or more microprocessors arranged on one or more electronic boards and using one or more algorithms, i.e. one or more computer programs, typically a microcontroller or similar. In other words, the operating unit 130 is configured to process and/or exploit the flow rate measurement signals or the flow rate values transmitted by the flow rate sensor 100.

Of course, the operating unit 130 can also be configured to operate other electromechanical elements integrated into the NO delivery apparatus 1, such as solenoid valves, or to control displays on a display screen (not shown) of the apparatus 1, such as a touch screen.

According to one embodiment, the operating unit 130 uses and/or comprises (at least) a prerecorded lookup table within storage means, such as a flash memory or the like, which makes it possible, depending on the flow rate measurement performed by the flow rate sensor 100, to calculate the quantity of NO to be added to the gas flowing in the inspiratory branch 31 in order to obtain the desired dosage of NO in the combined gas mixture that is to be administered to the patient P.

In other words, using the flow rate measurement(s) returned by the flow rate sensor 100 and the lookup table, the operating unit 130 can determine the flow rate of gas (e.g. air or O₂/N₂) from the mechanical ventilator 2 and the quantity of NO/N₂ mixture to be added, via the NO injection module 500, in order to obtain the desired NO concentration, i.e. the dosage defined by the physician, that is to be inhaled by the patient P. The quantity of NO to be supplied also depends on the NO concentration in the N₂/NO mixture from the one or more N₂/NO sources 250 feeding the NO delivery apparatus 1, as explained above.

More precisely, depending on the flow rate of gas (i.e. air or N₂/O₂) circulating in the inspiratory branch 31 and entering the upstream port 501 of the NO injection module 500, the operating unit 130 determines the quantity of NO, typically of NO/N₂ mixture, that must be injected by the NO injection module 500 into the inspiratory branch 31 of the patient circuit 3 in order to obtain the desired NO concentration in the combined gas mixture. The NO/N₂ mixture coming from the NO delivery apparatus 1 is supplied to the injection module 500 via an injection line 111 fluidically connecting the NO delivery apparatus 1 to the injection module 500, such as a flexible gas conduit or the like.

More precisely, the NO/N₂ mixture coming from the one or more N₂/NO sources 250, which is at a so-called “high” pressure since it can reach 140 to 200 bar abs, as explained above, feeds a high-pressure line 116 of the internal circuit of the apparatus 1, which line has a high-pressure inlet fluidically connected to the NO source(s) 250 in order to be supplied with NO/N₂ at high pressure.

A pressure regulator 115 arranged on the internal circuit of the apparatus 1 makes it possible to reduce the pressure of the NO/N₂ mixture (i.e. expanded gas) fed by the high-pressure line 116 to a predetermined value, for example about 4 bar abs or any other desired value. The outlet port of the pressure regulator 115 then supplies the NO/N₂ gas mixture to a low-pressure line 216 at the stable predetermined pressure, e.g. approximately 4 bar.

The expanded gas is collected and conveyed via the low-pressure line 216 of the internal circuit of the apparatus 1, which then feeds the injection line 111 which is itself fluidically connected to the injection module 500, via the NO injection channel 507.

The internal circuit of the NO delivery apparatus 1 comprises one or more gas passages or conduits, in particular the high-pressure and low-pressure lines 116, 216, and is arranged in the shell 10 of the apparatus 1.

A solenoid valve 113, for example a proportional solenoid valve, for example that of the miniature VSO series marketed by the company Parker®, is arranged on the internal circuit of the apparatus 1, typically on the low-pressure line 216, and is configured to control the flow rate of gaseous NO/N₂ supplied to the injection line 111. This gas flow rate is measured by an NO flow rate sensor 112, preferably arranged on the low-pressure line 216, downstream of the solenoid valve 113, as illustrated in FIG. 1.

The pressure regulator 115, the solenoid valve 113, the NO flow rate sensor 112 and the internal circuit comprising the high-pressure and low-pressure lines 116, 216 are arranged in the housing or shell 10 of the NO delivery device 1.

As has already been mentioned, the downstream end of the injection line 111 connects fluidically to the NO injection channel 507 of the injection module 500 in order to allow the NO/N₂ mixture to be injected into the injection module 500 and mix with the flow of gas containing oxygen (e.g. air) coming from the ventilator 2, which is conveyed via the upstream part of the inspiratory branch 31 of the patient circuit 3 and then enters the injection module 500 via its upstream port 501, and thus be able to obtain the combined gaseous mixture based on NO and oxygen.

The combined gaseous mixture obtained leaves the injection module 500 via the downstream port 502 thereof and is then conveyed to the respiratory interface 30 via the portion of the inspiratory branch 31 situated downstream of the injection module 500. In general, this combined gas mixture mainly comprises nitrogen (N₂), oxygen (O₂) in a content of at least 20 to 21 vol %, and NO in a content typically of between 1 and 80 ppmv, and optionally unavoidable impurities, including toxic NO₂ species resulting from untimely oxidation of NO. As is explained below, the injection module 500 of the invention makes it possible to minimize the formation of such toxic NO₂ species, by allowing rapid and efficient homogenization of the gas mixture produced in the injection module 500.

According to one embodiment, the inspiratory branch 31 can also comprise a humidifier (not shown) in order to humidify the gas delivered to the patient P, which humidifier is placed downstream of the NO injection module 500, that is to say between said NO injection module 500 and the respiratory interface 30 supplying the gas to the patient P.

In addition, the installation 1, 2 also generally comprises a gas sample sampling line (not shown in FIG. 1), which connects fluidically to the inspiratory branch 31, downstream of the NO injection module 500, that is to say between said NO injection module 500 and the respiratory interface 30 supplying the gas to the patient P, and furthermore to analysis means of the apparatus 1 (i.e. sensors for NO, O₂, NO₂, etc.), so that some of the combined gas mixture can be sampled and analysed in the apparatus 1 in order to ensure that its gaseous composition is in conformity with that expected, in particular that its NO content corresponds well to the desired dosage, that the oxygen content is well above at least 20 vol %, and that the harmful NO₂ impurities likely to be present remain below a given threshold, for example less than a few ppmv.

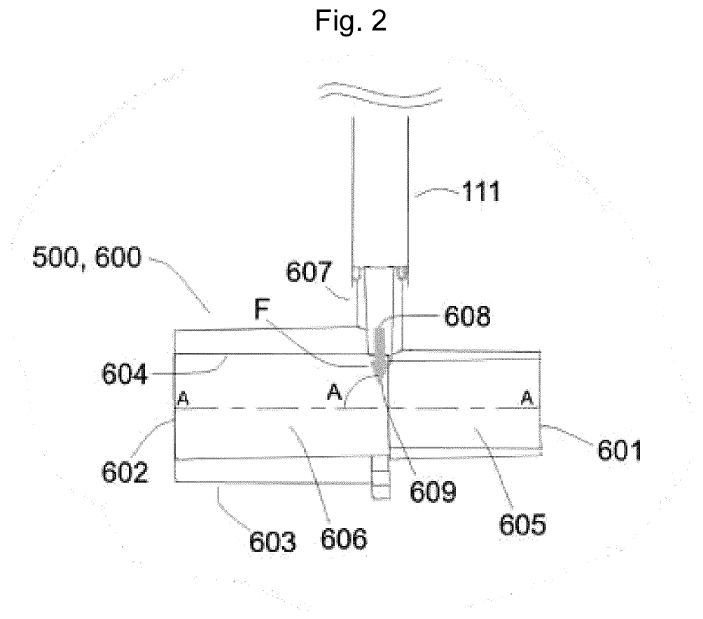

FIG. 2 is a sectional view of an embodiment of an injection module 500, 600 according to the prior art, which comprises a tubular body 603 of a cylindrical general shape, through which there extends a central passage 608 (i.e. a lumen) having a longitudinal axis (AA).

The tubular body 603 of the injection module 500, 600 comprises:
an upstream chamber 605 with an upstream port 601, also called a respiratory gas “inlet port or orifice”, intended to be fed with respiratory gas (O₂>20 vol %) coming from the ventilator 2.
a downstream chamber 606 with a downstream port 602, also called an “outlet port or orifice”, within which the flow of respiratory gas (O₂>20 vol %) coming from the ventilator 2 is mixed with the flow of NO/N₂ fed via the injection channel 607. The combined gas mixture thus obtained is then conveyed to the patient.
and an injection channel 607, cylindrical in shape, having an internal passage 608 (approximately 4 mm in diameter) connected substantially at the junction of the two chambers, upstream chamber 605 and downstream chamber 606, and opening into the central passage of the tubular body 603, that is to say approximately in the middle of the injection module 600.

The flow of respiratory gas (i.e. air or O₂/N₂) coming from the ventilator 2 circulates axially (along the axis AA) in the direction from the upstream chamber 605 to the downstream chamber 606.

The NO injection line 111 shown in FIG. 1 connects fluidically to the injection channel 607 in order to feed the internal passage 608 of the tubular body 603 with NO/N₂ mixture. The internal conduit 608 opens out at an injection port 609 arranged in the internal wall 604, that is to say a flush orifice formed in the internal wall 604 of the internal conduit 608, through which the NO/N₂ mixture flows (arrow F).

As can be seen, on account of the orientation of the internal conduit 608 substantially perpendicular to the internal wall 604 of the tubular body 603, and therefore also to the axis AA of the tubular body 603, the injection of the NO/N₂ mixture into the flow of respiratory gas is here perpendicular to the axis AA, that is to say at an angle of 90°. In other words, the flow of NO/N₂ mixture (symbolized by the arrow F) penetrates into the central passage 608 (lumen) of the tubular body 603 at an angle A of the order of 90° with respect to the longitudinal axis (AA) of the tubular body 603.

However, such an arrangement is not ideal, because the injection port 609 is located in a zone of low velocity of the respiratory gas coming from the mechanical ventilator 2, within the NO injection module 500, 600, and this does not allow efficient diffusion of the N₂/NO mixture.

Moreover, since the location of the injection port 609 is in a zone of low velocity, it appears that the O₂ molecules will migrate into the internal passage 608, or even into the injection line 111, depending on the velocity of the gas circulating respectively in the injection line 111 and in the internal conduit 608. The O₂ molecules present in the internal conduit 608, or even in the injection line 111, are then exposed to the NO/N₂ mixture conveyed via the injection line 111, which will promote the formation of NO₂, and the latter will then be in the downstream chamber 606 of the injection module 500, 600. This untimely oxidation of the NO is all the more considerable the higher the concentration of NO in the NO/N₂ mixture conveyed via the injection line 111.

In other words, the injection module 500, 600 of FIG. 2 does not allow rapid and homogeneous mixing of the gas flows, nor does it guarantee limited formation, preferably as little as possible, of NO₂.

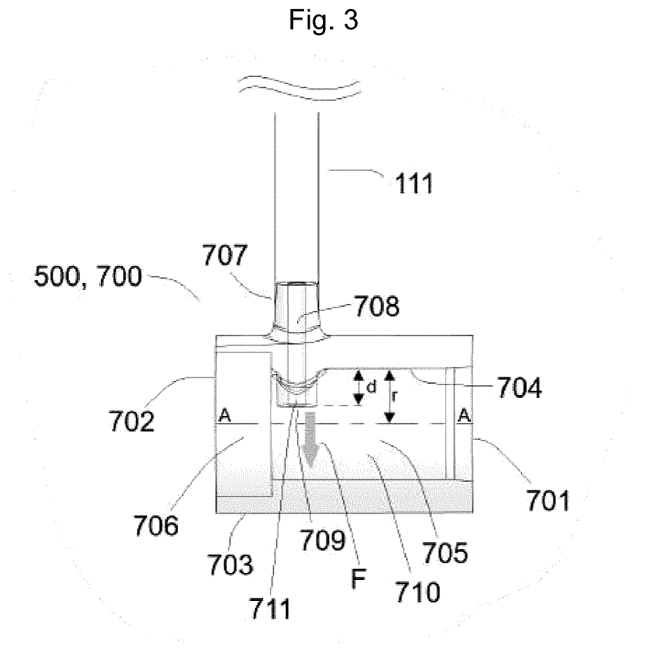

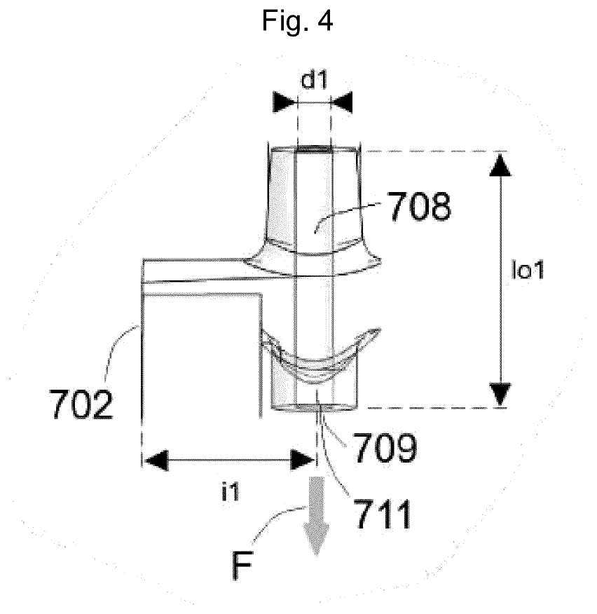

FIG. 3 and FIG. 4 are sectional views of another embodiment of an injection module 500, 700 of the prior art, as proposed for example by U.S. Pat. No. 10,894,140, which has a structure substantially similar to that of the injection module 500, 600 of FIG. 2.

Thus, the injection module 500, 700 again comprises a tubular body 703 of cylindrical shape, through which there extends an internal passage 710 (lumen) having a longitudinal axis (AA). The internal passage comprises an upstream chamber 705 comprising an upstream port 701 fed with respiratory gas from the mechanical ventilator 2; a downstream mixing chamber 706; and an injection channel 707 of cylindrical shape, having an internal passage or channel 708 of diameter d1 and of length lo1. The internal channel 708 is in fluidic communication with the injection line 111, which connects to said injection channel 707. The internal diameter d1 of the internal passage 708 is variable, typically between 0.1 mm and 1 mm, for example about 0.2 mm. Similarly, the length lo1 of the internal passage 708 is also variable, typically between 1 and 50 mm, for example about 10 mm.

The internal passage or channel 708 opens into an injection port 709. The centre of the injection port 709 of the internal channel 708 is situated at a distance i1 from the downstream port 702 of the injection module 700, as illustrated in FIG. 4, which is a magnified view of part of FIG. 3.

Here, however, the injection port 709 is not flush with the internal wall 704 of the hollowed-out envelope 703, as in the embodiment of FIG. 2, but is radially offset, that is to say situated close to the longitudinal axis (AA) of the tubular body 703.

More precisely, the injection port 709 is carried by a tubular expansion 711, which forms a nozzle or the like, projecting radially into the internal passage 710 (lumen) of the tubular body 703 in the direction of the axis (AA). For example, the distance d between the internal wall 704 of the tubular body 703 and the free end of the tubular expansion 711 carrying the injection port 709 can be of the order of 60% of the internal radius r of the tubular body 703, which is cylindrical in shape.

Here again, the flow of respiratory gas (i.e. air or O₂/N₂) coming from the ventilator 2 circulates axially (along the axis AA) in the direction from the upstream chamber 605 to the downstream chamber 606.

Furthermore, it will be noted that, as in the embodiment of FIG. 2, the injection of the flow of NO/N₂ is effected in a (quasi) perpendicular direction (arrow F). Such an arrangement aims to take advantage of the distribution of the velocity profile of the gas circulating axially (axis AA) in the cylindrical internal passage 710 (lumen) of the tubular body 703, which is maximum at the centre, i.e. at the level of the axis AA, and on average (almost) zero at the level of the internal wall 704.

However, obtaining a homogeneous mixture of the NO-containing gas and of the respiratory gas coming from the mechanical ventilator 2 (i.e. N₂/O₂ mixture) depends on the velocity of these gases. The higher this velocity, the shorter the time for which the gas mixture is considered homogeneous.

Although the injection module 500, 700 of FIG. 3 and FIG. 4 leads to better results than the injection module 500, 600 of FIG. 2, through the use of a nozzle or the like projecting radially into the gas passage carrying the flow from the ventilator 2, and distributing the N₂/O₂ mixture at a given distance from the axis AA, in order to allow time for the N₂/O₂ mixture leaving injection port 709 to expand before mixing with the respiratory gas (i.e. air or O₂/N₂) in the regions situated near the axis AA, where the flow velocity is maximum, it has been found in practice that this configuration is also not ideal.

Thus, it does not completely eliminate the phenomenon of diffusion of the O₂ in the internal conduit 708, because of the perpendicular nature of the injection of the NO-containing gas into the respiratory gas coming from the mechanical ventilator 2. Indeed, in the vicinity of injection port 709, O₂ is still able to migrate into the internal conduit 708 and there form toxic NO₂ species in contact with NO molecules, in particular when the N₂/O₂ mixture used contains a high concentration of NO, i.e. more than 1000 ppmv.

Moreover, the transverse injection of the N₂/NO mixture, ejecting at high velocity from the injection port 708, will form a gaseous “plume” in contact with the N₂/O₂ mixture, leading to concentration of a not inconsiderable part of the injected N₂/NO mixture, and therefore of the NO molecules, in zones of low velocity, in particular in the vicinity of the internal wall 704, thereby creating a temporary imbalance in the distribution of the gas in the downstream chamber 706, which leads to an increase in the propensity to form NO₂ by increasing the time necessary for obtaining a homogeneous gas mixture.

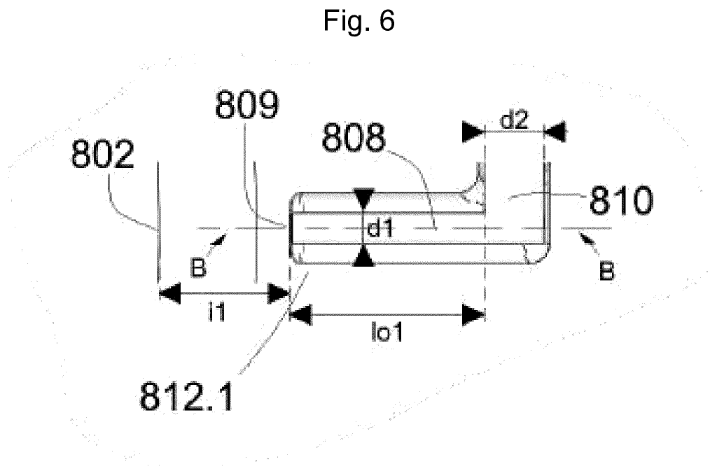

FIG. 5 and FIG. 6 show schematically an embodiment of an injection module 500, 800 according to the present invention, usable in an NO supply installation 1, 2, 3, such as the one in FIG. 1 or a similar installation.

This injection module 500, 800 is designed to permit injection of NO/N₂ mixture having “conventional” (i.e. <1000 ppmv) and also “high” (i.e. >1000 ppmv) NO contents into the flow of respiratory gas coming from the ventilator 2 and to make it possible to obtain homogeneous mixtures while minimizing the formation of NO₂.

The injection module 500, 800 has a general shape similar to those of the preceding modules of FIG. 2 to FIG. 4 in that it comprises a tubular body 803 of cylindrical shape, through which there extends an internal passage (i.e. a lumen) and a longitudinal axis (AA). The tubular body 803 comprises an upstream chamber 805 which receives the respiratory gas (i.e. air or O₂/N₂ mixture) delivered by the ventilator 2 via an upstream port 801 (i.e. inlet orifice) of the injection module 800, and a downstream mixing chamber 806 where the mixing between the flow of NO/N₂ and the flow of respiratory gas from the mechanical ventilator 2 will take place in order to obtain the combined homogeneous mixture containing NO at the desired dosage.

The tubular body 803 is preferably made of polymer.

The downstream mixing chamber 806 comprises a downstream port 802, that is to say an outlet orifice, via which the combined gaseous mixture leaves the tubular body 803 before being conveyed by the downstream part of the inspiratory branch 31 of the patient circuit 3 as far as the respiratory interface 30 supplying the patient P, as has already been explained.

As previously, the flow of respiratory gas (containing >20% O₂) from the mechanical ventilator 2 circulates axially (i.e. along the axis AA) from the upstream chamber 805 to the downstream mixing chamber 806, that is to say in the direction from the upstream port 801 to the downstream port 802.

Here again, the tubular body 803 of the injection module 500, 800 comprises an injection channel 807 through which there extends an upstream internal passage 813 of diameter d2, to which is connected the injection line 111 feeding the NO/N₂ mixture delivered by the NO delivery apparatus 1.

As in the previous embodiment and illustrated in FIG. 5, the injection port 809 fed in particular by the injection channel 807, through which the NO/N₂ mixture enters the internal passage 811 (lumen) of the tubular body 803, is carried by a tubular expansion 812, advantageously constituting or forming a nozzle or the like, projecting into the internal passage 811 (lumen) of the tubular body 803 in the direction of the axis (AA).

However, in contrast to the embodiment of FIG. 3 and FIG. 4, the tubular expansion 812 is not rectilinear but, according to the invention, has an angled, i.e. curved shape, namely what is substantially an L shape.

The angled tubular expansion 812 has a first internal conduit section 810 of diameter d2 and a second internal conduit section 808 of diameter d1 and length lo1. The first internal conduit section 810 is situated between the upstream internal passage 813 and the second internal conduit section 808, as illustrated in FIG. 5 and FIG. 6, which is a magnified view of the tubular expansion 812 of FIG. 5.

In other words, the upstream internal passage 813, the first internal conduit section 810 and the second internal conduit section 808 are in fluidic communication with each other, so that the flow of NO/N₂ fed via the injection line 111 circulates successively in these passages/sections before leaving them via the injection port 809, which is located at the free end 812.1 of the second internal conduit section 808.

The diameter d2 of the upstream internal passage 813 and/or of the first internal conduit section 810 can be between 1 and 5 mm, for example of the order of 3 mm. In all cases, it is greater than the diameter d1 of the second internal conduit section 808 of the angled tubular expansion 812, i.e. d2>d1.

As can be seen, the axis BB of the second internal conduit section 808 of the angled tubular expansion 812, such as a nozzle or the like, also referred to hereinafter as the “angled nozzle”, is oriented parallel to the longitudinal axis AA of the tubular body 803 of the injection module 500, 800, so as to inject the NO/N₂ mixture in the same direction (i.e. along the axis AA) as the flow of respiratory gas circulating in the internal passage 811 (lumen) of the tubular body 803, namely the direction of the arrow F in FIG. 5.

Advantageously, they are coaxial, as illustrated in FIG. 5 and FIG. 6, that is to say that the axes AA and BB of the second internal conduit section 808 of the angled nozzle 812 and of the tubular body 803 are coincident.

In other words, in the embodiment of FIG. 5 and FIG. 6, the second internal conduit section 808 of the angled tubular expansion 812, i.e. of the angled nozzle, is centred on the longitudinal axis AA of the tubular body 803, i.e. arranged coaxially to the longitudinal axis AA, so that the flow of NO exiting via the injection port 809 of the angled nozzle 812 is injected into the centre of the tubular body 803 (axis AA) and in the same direction as the flow of respiratory gas circulating in the internal passage 811 (lumen) of the tubular body 803, that is to say in the direction of the downstream mixing chamber 806 and its downstream port 802.

As is shown schematically in FIG. 6, the second internal channel section 808 comprises an internal diameter d1 which can be between 0.1 and 1 mm, for example of the order of 0.2 mm, and a length lo1 which can be between 1 and 50 mm, for example about 10 mm.

The second internal channel section 808 of the angled nozzle 812 terminates in the injection port 809, which is also centred on the axis AA and located at a distance i1 from the downstream port 802 of the tubular body 803 of the injection module 500, 800. The distance i1 can be between 1 and 10 mm for example.

As can be seen, the injection port 809 of the second internal channel section 808 of the angled nozzle 812 faces the downstream port 802 of the injection module 500, 800, i.e. they are arranged facing each other.

In this advantageous configuration according to the invention, the injection of the flow of NO/N₂ mixture takes place in parallel and in the same direction, preferably coaxially, as the flow of respiratory gas containing at least 20 vol % of oxygen (i.e. air or O₂/N₂ mixture) supplied by the mechanical ventilator 2 and circulating in the internal passage 811 (lumen) of the tubular body 803, that is to say in the direction from the upstream port 801 to the downstream port 802 of the injection module 800.

Thus, the tubular expansion or angled nozzle 812 is directed or oriented so as to deliver the flow of NO/N₂ mixture in parallel and in the direction of the flow of respiratory gas coming from the ventilator 2 and circulating in the lumen 811 of the injection module 500, 800, preferably coaxially with respect to said flow of respiratory gas, as has already been explained.

Situated downstream of the injection port 809 is the downstream mixing chamber 806 in which the mixing between the flow of NO/N₂ and the flow of respiratory gas from the mechanical ventilator 2 will take place in order to give the desired homogeneous mixture containing the desired/adjusted dosage of NO, for example an NO concentration of between 1 and 80 ppmv.

According to one configuration of the invention, the fact that the axes AA and BB are preferably coaxial, and therefore that the injection port 809 of the angled nozzle is situated on the axis AA of the tubular body 803 of the injection module 500, 800, the mixing of the flow of NO/N₂ with that of the respiratory gas (i.e. air or O₂/N₂ mixture with an O₂ content of >20 vol %) coming from the ventilator 1 and entering the injection module 500, 800 via its upstream port 801 is carried out efficiently, because the velocity of the gas circulating in the cylindrical conduit is maximum at the level of the axis AA, which accelerates the mixing of the flows of gas with each other.

This results in rapid formation of a homogeneous mixture, whereas, conversely, the formation of undesirable NO₂ species through NO oxidation with oxygen present in the respiratory gas is limited, in particular by comparison with the previous injection module 500, 700, and this even in the presence of a (very) high concentration of NO in the N₂/NO mixture, typically greater than 1000 ppmv, for example between 2000 and 20000 ppmv.

By virtue of the particular configuration of the injection module 500, 800 according to the invention in FIG. 5 and FIG. 6, the diffusion of O₂ into the second internal conduit 808 through the injection port 809 is reduced on account of the common direction of circulation of the gas flows and the particular orientation of the angled nozzle 812 of the injection module 500, 800 according to the invention. Indeed, it is much more difficult for the oxygen molecules to oppose the general directional movement, that is to say to ascend in a “counter-current” in order to migrate into the second internal conduit 808.

More generally, the particular configuration of the injection module 500, 800 according to the invention in FIG. 5 and FIG. 6 has the effect that the high-velocity injection of gas, i.e. the flow of NO, will spread more uniformly, so the different zones of the downstream chamber 806 receive a substantially identical quantity of injected N₂/NO mixture, thus generating a distribution equilibrium making it possible to limit the formation of undesirable NO₂ species.

In order to show the effectiveness of an injection module 500, 800 according to the invention in FIG. 5 and FIG. 6, comparative simulation tests were carried out using the various injection modules 500 illustrated in FIG. 2 to FIG. 6, in order to evaluate the diffusion of O₂ molecules in the internal conduits 608, 708 and 808 of these various NO injection modules 600, 700, 800, 500.

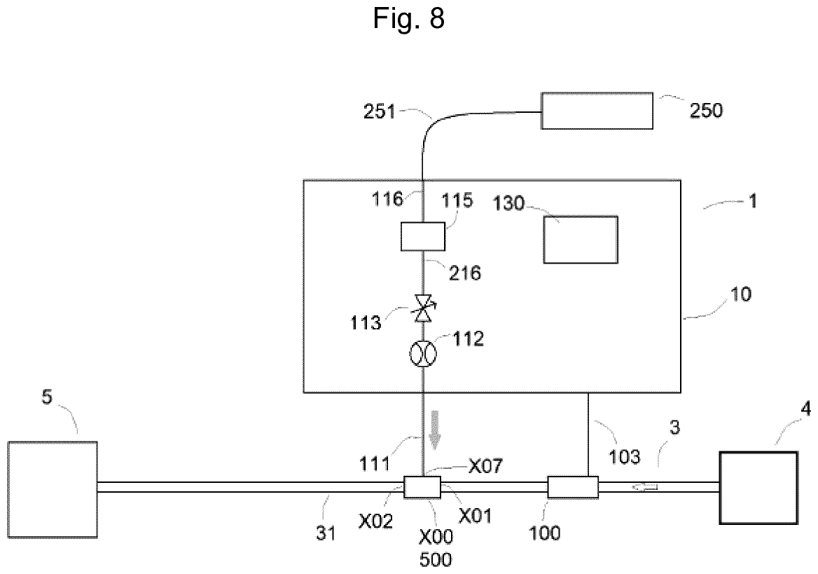

The simulation test conditions are as follows:
cylinder containing N₂/NO with a high content of NO, here 20,000 ppmv of NO.
flow rate of 5 l/min of pure oxygen coming from the mechanical ventilator 2.
adjusted dosage: 80 ppmv of NO in the combined gas sent to the patient.
the experimental set-up used to carry out the tests is illustrated in FIG. 8 and detailed below.

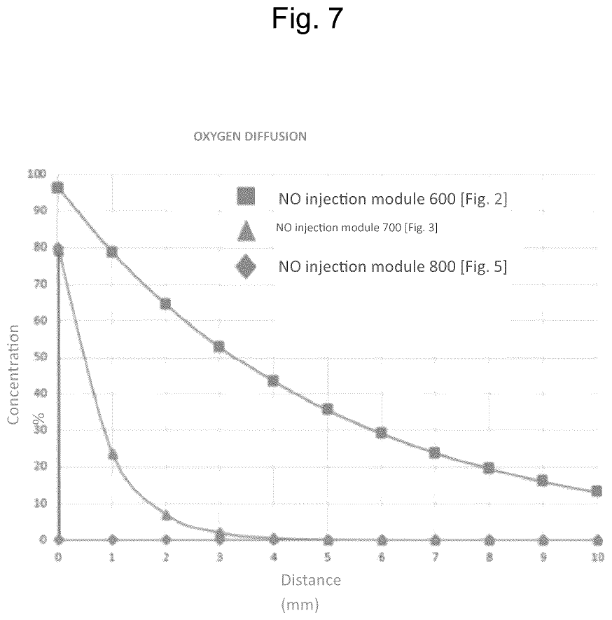

The results obtained were recorded in FIG. 7, which is a graph illustrating the diffusion of oxygen molecules. The x-axis corresponds to the distances (in mm) and the y-axis gives O₂ concentration values (in vol %).

The origin 0 of the x-axis corresponds to the various injection ports 609, 709 and 809 of the injection modules 600, 700 and 800, that is to say that the distance of “0 mm” is the reference point corresponding to the location of the various injection ports 609, 709 and 809. A positive distance, for example 2 mm, corresponds to the distance separating the injection ports 609, 709 and 809 from a “point” of each internal conduit 608, 708 and 808 situated 2 mm from the injection port 609, 709 and 809 in question. Similarly, another positive distance, for example 5 mm, corresponds to the distance separating the injection ports 609, 709 and 809 from a “point” of the internal conduits 608, 708 and 808 situated 5 mm from the injection ports 609, 709 and 809.

It appears that the NO injection module 500, 600 of the prior art in FIG. 2, under the above-mentioned conditions (i.e. 20000 ppmv of NO), sees the O₂ molecules massively diffuse into its internal conduit 608. At its injection port 609, the O₂ concentration is close to 100%; at a distance of 3 mm, this concentration is of the order of 50%, and 15% at a distance of about 10 mm. This is mainly due to the low velocity of the gas circulating in the internal conduit 608, due to the relatively large diameter of the internal conduit 608, and to the method of flush perpendicular injection, as has already been explained.

With the NO injection module 500, 700 of the prior art in FIG. 3 and FIG. 4, under the same conditions, the O₂ molecules diffuse less in its internal conduit 708. At the injection port 709, the O₂ concentration is close to 80%. This is due to the higher-velocity ejection of the N₂/NO mixture at the injection port 709. At a distance of 1 mm, this concentration is of the order of 25% and is close to 0% at a distance of 3 mm. The high velocity of the gas circulating in the internal conduit 708, of much smaller diameter than the internal conduit 608 of the preceding embodiment, limits the progression of the diffusion of O₂. However, untimely diffusion of the O₂ molecules still occurs, which inevitably leads to the formation of harmful NO₂ species.

Finally, with an NO injection module 800 according to the invention, as illustrated in FIG. 5 and FIG. 6, using an angled nozzle 812 configured to inject the NO/N₂ flow centred (i.e. coaxial with respect to the AA axis) and in the direction of circulation of the respiratory gas coming from the mechanical ventilator 2, there is no diffusion of O₂ molecules in the internal conduit 808. In fact, an 02 concentration of 80% is present only at the injection port 809 and drops to 0% inside the internal conduit 808.

In this case, untimely diffusion of the O₂ molecules cannot occur, and therefore any formation of harmful NO₂ species will be rendered impossible.

As is illustrated in FIG. 8, the experimental set-up used for measuring the performance of the various injection modules tested, namely those according to the prior art in FIG. 2 to FIG. 4 and the one according to the invention in FIG. 5 and FIG. 6, is generally analogous to the installation of FIG. 1 described above, in particular as regards the NO delivery apparatus 1 and its operation, except that in this experimental set-up:
the patient circuit 3 of the installation in FIG. 1 has been simplified by omission of the expiratory branch 32.
the mechanical ventilator 2 of FIG. 1 has been replaced by an O₂ flow generator 4, making it possible to deliver a constant flow of O₂ in the inspiratory branch 31.
the patient has been replaced by a gas analyser 5 for measuring the concentration of NO₂ in the gas mixture that it receives and analyses, for example the analyser designated FTIR Multigas 2030 available from MKS® or the analyser designated 42iQHL available from Thermo Fisher Scientific®.

In FIG. 8, the NO injection module X00, 500, having upstream port X01 and downstream port X02, as well as an injection channel X07, has been one or other of the NO injection modules 600, 700, 800, 500 described above, depending on the test performed, i.e. X can take the value of 6, 7 or 8 depending on the NO injection module tested.

In addition, the gas analyser 5 is situated at a distance of approximately 1 m from the injection module X00, 500.

The NO₂ values measured by the gas analyser 5 with the different configurations of NO injection modules 600, 700, 800, 500 described above for an NO concentration of 20 000 ppmv, as explained above, are set out in Table 1 below.

TABLE 1

| O₂ flow rate (l/min) | | 1 | 5 | 20 |
|---|---|---|---|---|
| NO₂ | Module 600 | 34 | 12.2 | 7.6 |
| (ppm) | Module 700 | 11 | 5.3 | 4.1 |
| | Module 800 | 10.8 | 5.1 | 3.9 |

Other comparative tests were carried out with an NO concentration of 5000 ppm in the NO/N₂ mixture. However, in this case, in order to maintain a similar velocity of gas circulating in the internal conduits 708, 808 of the NO injection modules 700, 800, the diameter d1 of said internal conduits 708, 808 was brought to approximately 0.4 mm.

Under these conditions, the NO₂ values measured by the gas analyser 5 for the different configurations of NO injection modules 600, 700, 800, 500 described above for an NO concentration of 5000 ppmv are set out in Table 2 below.

TABLE 2

| O₂ flow rate (l/min) | | 1 | 5 | 20 |
|---|---|---|---|---|
| NO₂ | Module 600 | 6 | 2.7 | 2 |
| (ppm) | Module 700 | 5.1 | 2.4 | 1.9 |
| | Module 800 | 4.9 | 2.2 | 1.8 |

These tests show that, regardless of the NO concentration (i.e. 5000 or 20000 ppmv), the NO injection modules 700, 800 of FIG. 3 to FIG. 6 make it possible to significantly reduce the concentration of NO₂ formed compared to the NO injection module 600 of FIG. 2. This demonstrates the value of using a tubular expansion, such as an injection nozzle or the like, projecting into the internal passage (lumen) of the tubular body.

However, the configuration of the NO injection module 500, 800 according to the invention using a curved tubular expansion 812 oriented to allow the injection of the N₂/NO flow to be effected in parallel, and in the same direction as the gas mixture coming from the mechanical ventilator 2 (in this case, O₂ coming from O₂ flow generator 4 during the tests) makes it possible to further reduce the formation of NO₂ compared to the NO injection module 500, 700 of the prior art according to FIG. 3 and FIG. 4 which proposes using a rectilinear nozzle oriented toward the axis AA, on account of less diffusion of O₂ in the internal conduit 808 of the NO injection module 500, 800 according to the invention, as well as more rapid homogenization of the N₂/NO gas mixtures and for example N₂/O₂ or air mixtures in its downstream chamber 806.

In general, the installation of the invention is particularly well suited to the treatment of persons, i.e. a human patient (i.e. adults, children, adolescents or neonates), suffering from pulmonary hypertension and/or hypoxia, generating pulmonary vasoconstrictions or the like, which result from one or more pathologies or other pulmonary disorders typically of the PPHN type (persistent pulmonary hypertension in the newborn) or ARDS (acute respiratory distress syndrome), or may occur during cardiac surgery with extracorporeal blood circulation (ECC).

What is claimed is:

1. Gas supply installation (1, 2, 3) comprising a gas injection module (500, 800), to which there are connected:
a mechanical ventilator (2) supplying a flow of respiratory gas containing at least 20 vol % of oxygen, and
an NO delivery apparatus (1) supplying a flow of NO/N₂ gas mixture,
said gas injection module (500, 800) comprising a tubular module body (803) through which there extends an internal passage (811) comprising a longitudinal axis (AA),
said tubular module body (803) comprising:
an upstream port (801) through which the flow of respiratory gas can enter the internal passage (811),
a downstream port (802) through which a combined gas flow can exit the internal passage (811),
an external injection conduit (807) fluidically connecting to the tubular module body (803) and comprising an internal passage (813) for conveying the flow of NO/N₂ gas mixture, and
a tubular expansion (812) forming an injection nozzle arranged in the internal passage (811) of the tubular body (803) and comprising an internal nozzle passage (808) in fluidic communication with the internal passage (813) of the injection conduit (807), said tubular expansion (812) further comprising an injection port (809) arranged at a free end (812.1) of said tubular expansion (812), said injection port (809) opening into the internal passage (811) of the tubular module body (803),
characterized in that the tubular expansion (812) of the tubular module body (803) of the injection module (500, 800) has a generally curved or angled shape and comprises at least one section carrying the injection port (809), arranged parallel to the longitudinal axis (AA) and directed towards the downstream port (802).

2. Installation according to claim 1, characterized in that the internal passage (811) of the tubular module body (803) of the injection module (500, 800) comprises an upstream chamber (805) comprising the upstream port (801) and/or a downstream mixing chamber (806) comprising the downstream port (802).

3. Installation according to claim 1, characterized in that the tubular expansion (812) of the tubular module body (803) of the injection module (500, 800) has a general “L” shape.

4. Installation according to claim 1, characterized in that the section (808) carrying the injection port (809) of the tubular expansion (812) of the tubular module body (803) of the injection module (500, 800) is arranged coaxially with respect to the longitudinal axis (AA) of the internal passage (811) of the tubular module body (803).

5. Installation according to claim 1, characterized in that the tubular expansion (812) of the tubular module body (803) of the injection module (500, 800) comprises a first conduit section (810) and a second conduit section (808), wherein:
the second conduit section (808) comprises the injection port (809) and the free end (812.1) of said tubular expansion (812), and
the first conduit section (810) is arranged between the second conduit section (808) and the internal passage (813) of the injection conduit (807) and in fluidic communication with the second conduit section (808) and the internal passage (813).

6. Installation according to claim 1, characterized in that the external injection conduit (807) of the tubular module body (803) of the injection module (500, 800) connects to the tubular peripheral wall of the tubular module body (803).

7. Installation according to claim 1, characterized in that the tubular module body (803) of the injection module (500, 800) is configured, at the upstream port (801) and the downstream port (802), to permit a fluidic connection of gas conduits forming at least a part of the inspiratory branch (31) of the patient circuit (3).

8. Installation according to claim 1, characterized in that the NO delivery apparatus (1) is fluidically connected and fed with an NO/N₂ mixture by one or more gas containers (250) containing an NO/N₂ mixture.

9. Installation according to claim 1, characterized in that the NO delivery apparatus (1) is fed with an NO/N₂ mixture containing between 100 and 20,000 ppmv of NO, the remainder being nitrogen (N₂).

10. Installation according to claim 1, characterized in that the injection module (500, 800) is arranged in an inspiratory branch (31) of a patient circuit (3), said inspiratory branch (31) being fed by the mechanical ventilator (2) with respiratory gas containing at least 20 vol % of oxygen.

11. Installation according to claim 1, characterized in that the tubular expansion (812) of the tubular module body (803) forms a curved nozzle.

12. Installation according to claim 1, characterized in that the tubular expansion (812) of the gas injection module (500, 800) is oriented for injecting the NO/N₂ mixture into the internal passage (811) of longitudinal axis (AA) in the same direction as the flow of respiratory gas containing at least 20 vol % of oxygen circulating in the internal passage (811).

13. Installation according to claim 1, characterized in that the NO delivery apparatus (1) comprises operating means, an internal gas circuit, valve means arranged on the internal gas circuit for controlling the flow of NO/N₂ in the internal gas circuit, and at least one NO outlet for supplying the NO/N₂ gas flow.

14. Installation according to claim 1, characterized in that a flow rate sensor is arranged in the patient circuit (3) between the ventilator (2) and said gas injection module (500, 800).

15. Installation according to claim 1, characterized in that the mechanical ventilator (2) is configured to supply a flow of respiratory gas containing at least 20 vol % of oxygen, chosen from air or an O₂/N₂ mixture.
